SEQUENCE LISTING (30 sequences)

SEQ ID NO: 1 - DNA, 21 bp, Artificial Sequence
  PCR Primer
acccaaaaggaaagaaaaacc

SEQ ID NO: 2 - DNA, 21 bp, Artificial Sequence
  PCR Primer
gtagagcagtgtgagcaggaa

SEQ ID NO: 3 - DNA, 21 bp, Artificial Sequence
  PCR Primer
gtgtttgtgtggaagactgga

SEQ ID NO: 4 - DNA, 21 bp, Artificial Sequence
  PCR Primer
aggaggaagaggacgatgaag

SEQ ID NO: 5 - DNA, 21 bp, Artificial Sequence
  PCR Primer
gacaagatgaagacccacctg

SEQ ID NO: 6 - DNA, 21 bp, Artificial Sequence
  PCR Primer
ttgctctgggtggcagtaaca

SEQ ID NO: 7 - DNA, 23 bp, Artificial Sequence
  PCR Primer
agagaaaaattacccgactcctc

SEQ ID NO: 8 - DNA, 23 bp, Artificial Sequence
  PCR Primer
catccctcttgcgtttgttggtg

SEQ ID NO: 9 - DNA, 20 bp, Artificial Sequence
  PCR Primer
gggggctataagttctttgc

SEQ ID NO: 10 - DNA, 20 bp, Artificial Sequence
  PCR Primer
tccaacacttcgagaggtcc

SEQ ID NO: 11 - DNA, 21 bp, Artificial Sequence
  PCR Primer
gcccgtggagattgagaggac

SEQ ID NO: 12 - DNA, 21 bp, Artificial Sequence
  PCR Primer
gtgcttggagttattgtggac

SEQ ID NO: 13 - DNA, 21 bp, Artificial Sequence
  PCR Primer
tcccccagaggtcagtgttac

SEQ ID NO: 14 - DNA, 21 bp, Artificial Sequence
  PCR Primer
gccagtccaaagtccccaatc

SEQ ID NO: 15 - DNA, 29 bp, Artificial Sequence
  PCR Primer
gcggaattcccagtcatgaagaagatgca

SEQ ID NO: 16 - DNA, 30 bp, Artificial Sequence
  PCR Primer
gcgggatccgcagcacttgagtaggttcac

SEQ ID NO: 17 - DNA, 28 bp, Artificial Sequence
  PCR Primer
gcggaattccaccatccctgacggtgaa

SEQ ID NO: 18 - DNA, 30 bp, Artificial Sequence
  PCR Primer
gcgggatccgggggtgacactaacgaggat

SEQ ID NO: 19 - DNA, 30 bp, Artificial Sequence
  PCR Primer
gcggaattcctgtggcacggaacttctagt

SEQ ID NO: 20 - DNA, 30 bp, Artificial Sequence
  PCR Primer
gcgaagcttcctgtccgaagagtcactatg

SEQ ID NO: 21 - DNA, 21 bp, Artificial Sequence
  PCR Primer
cctgggcatctgaggactttt

SEQ ID NO: 22 - DNA, 21 bp, Artificial Sequence
  PCR Primer
ggggatactgaggctgcttga

SEQ ID NO: 23 - DNA, 21 bp, Artificial Sequence
  PCR Primer
agtggcaaaggaggtagagat

SEQ ID NO: 24 - DNA, 21 bp, Artificial Sequence
  PCR Primer
aacatagggaagcaacagaat

SEQ ID NO: 25 - DNA, 21 bp, Artificial Sequence
  PCR Primer
caaaagaagtccagagcagtc

SEQ ID NO: 26 - DNA, 21 bp, Artificial Sequence
  PCR Primer
gggcaaaatagggcatcagtt

SEQ ID NO: 27 - DNA, 21 bp, Artificial Sequence
  PCR Primer
gtgggcaatcagagtcgtcaa

SEQ ID NO: 28 - DNA, 21 bp, Artificial Sequence
  PCR Primer
atgtgggagtgctgtggagat

SEQ ID NO: 29 - DNA, 24 bp, Artificial Sequence
  PCR Primer
catggcgagaagctctttagttcc

SEQ ID NO: 30 - DNA, 24 bp, Artificial Sequence
  PCR Primer
tgcaaggcgattaagttgggtaac

(19) United States
(12) Patent Application Publication
(10) Pub. No.: US 2004/0029271 A1
(43) Pub. Date: Feb. 12, 2004

(54) Pax5-deficient pro-B cells, methods of producing them and the use of such cells in human therapy

(75) Inventors: Meinrad Busslinger, Vienna (AT); Ingvild Mikkola, Kvaloysletta (NO); Barry Heavey, Athlone (IE)

Correspondence Address: STERNE, KESSLER, GOLDSTEIN & FOX PLLC, 1100 NEW YORK AVENUE, N.W., WASHINGTON, DC, 20005, US

(73) Assignee: Boehringer Ingelheim International GmbH

(21) Appl. No.: 10/431,599

(22) Filed: May 8, 2003

Related U.S. Application Data
(60) Provisional application No. 60/385,582, filed on Jun. 5, 2002.

(30) Foreign Application Priority Data
May 8, 2002 (EP) 02 010 439.4

(51) Int. Cl. C12N 5/08

(52) U.S. Cl. 435/372

(57) ABSTRACT

Human pro-B cells deficient in Pax5 expression and methods of producing them. Human Pax5-deficient pro-B cells are useful for the therapy of disorders associated with a depletion of the lymphoid system, in particular for the treatment of immunodeficiencies like AIDS.

### CROSS REFERENCE TO RELATED APPLICATIONS

This application claims the benefit of U.S. Provisional Appl. No. 60/385,582, filed Jun. 5, 2002, and European Appl. No. EP 02 010 439.4, filed May 8, 2002, both of which are herein incorporated by reference.

### BACKGROUND OF THE INVENTION

1. Field of the Invention

The present invention relates to the field of immunology, in particular to the therapy of disorders associated with defects in the lymphoid system, e.g. immunodeficiencies.

2. Description of Related Art

The hematopoietic stem cell gives rise to all lymphoid lineages by differentiating through the intermediary stage of the common lymphoid progenitor (Kondo et al., 1997). Entry of this progenitor into the B cell pathway depends on the transcription factors E2A, EBF and Pax5 (BSAP), which control two separate transcriptional programs (Busslinger et al., 2000). The regulators E2A and EBF coordinately activate the expression of B-cell-specific genes (Sigvardsson et al., 1997; Kee and Murre, 1998), which is essential for the generation of the earliest B cell progenitors (pro-B cells) at the onset of B-lymphopoiesis (Zhuang et al., 1994; Bain et al., 1994; Lin and Grosschedl, 1995). The mere activation of the B-lymphoid expression program is, however, not sufficient for commitment to the B cell lineage, as Pax5-deficient pro-B cells still retain the broad developmental potential of early hematopoietic progenitors (Rolink et al., 1999; Nutt et al, 1999a) despite normal expression of E2A and EBF (Nutt et al., 1997). Instead, Pax5 is essential to repress the promiscuous transcription of lineage-inappropriate genes and to commit progenitor cells to the B cell pathway by suppressing alternative cell fates (Rolink et al., 1999; Nutt et al, 1999a).

Recently, the inventors demonstrated that Pax5 is also essential for maintaining the identity of mature B cells in late B-lymphopoiesis (Horcher et al., 2001). In response to Pax5 inactivation, mature B cells down-regulated the transcription of many B-cell-specific genes and simultaneously activated the expression of non-B-lymphoid genes (Horcher et al., 2001). However, lineage-tracing experiments failed so far to provide evidence that the mature Pax5-deficient B cells became decommitted and differentiated in vivo into other hematopoietic cell types.

Whether the commitment of tissue-specific stem cells to different developmental pathways is a reversible or irreversible process is a fundamental question in the field of organogenesis (Weissman, 2000). This question has recently been investigated for the neural crest stem cell that generates either neurons or glial cells during peripheral nervous system development. In this case, transient activation of Notch was shown to be sufficient to irreversibly commit neural crest stem cells to the glial cell fate (Morrison et al., 2000). The question of reversibility is also of high importance in view of hematologically-based therapies of disorders that are accompanied by immunodeficiencies.

Fundamental to the pathophysiology of the acquired immunodeficiency syndrome (AIDS) is the inability of the immune system to compensate for the depletion of specific immune effector cells caused by HIV-1 (HIV). By targeting the cells responding to it, HIV undermines the immune response favoring viral spread in a self-accelerating manner. Yet, for some individuals the immune system remains vigorous and capable of controlling HIV (Rosenberg et al., 1997).

Anti-retroviral medication can diminish the rapidity of viral spread but does not appear able to restore critical, virus-controlling immunity. Immune recovery observed with anti-retroviral drugs is not sufficient to permit immune control of HIV without medications. Strategies to overcome this problem and to regenerate vigorous HIV-specific immunity focus on two basic goals: 1) To provide additional anti-HIV protection to developing cells; or 2) To enhance the generation of specific T cell subsets. Currently, potent new anti-viral drugs and vaccines are regarded as leading methods to achieve these goals. Autologous cells manipulated ex vivo and adoptively provided to alter the balance in favor of host control of HIV is considered a plausible alternative (Levine et al., 2001).

A number of efforts are ongoing using retroviral transduction of primitive cells. By transduction of genetic constructs to protect cells from HIV infection, enhanced immune reconstitution is hypothesized and currently being tested.

Enhancing T-lymphoid regeneration is an obviously desirable goal that stem cell expansion strategies may ultimately be tailored to achieve. In addition, ex vivo T cell differentiation systems are being developed (Levine et al., 2001).

The ability to achieve T cell neogenesis ex vivo has been difficult except by using organ culture or, with limited success, co-culture systems. There are currently other efforts using three-dimensional matrices that permit single positive CD4+and CD8+cells to emerge from CD34+or AC133+bone marrow cells (Poznansky et al., 2000). While de novo T cell generation has been documented in these systems, the ability to expand this system to a clinical scale is unlikely and so far untested.

Alternative strategies to achieve improved immune function using ex vivo T cell manipulation are also being tested. One such method involves the ex vivo expansion of existing circulating T cells in HIV individuals using a method that results in a HIV-free population of CD4+and CD8+cells. (Levine et al., 1996). A modification of this approach is to transduce the cells during the expansion phase with a chimeric T cell receptor gene (Yang et al., 1997). Clinical studies using this approach have demonstrated successful transduction and survival of the transduced T cells in vivo for up to 6 months (Mitsuyasu et al., 2000).

The above summary shows that immune regeneration is only partially successful with available anti-retroviral strategies, providing protection from most opportunistic infections but failing to achieve the immune control of HIV that is now considered to be possible. Achieving immune control without the need for chronic anti-HIV medications is clearly a goal of enormous value and will require regeneration of the robust HIV-specific immune response seen with acute HIV infection. Further definition of events restricting the immune regeneration of this response and strategies to overcome these restrictions are an ongoing challenge with tremendous therapeutic potential. Hematologically based therapies using gene-modified cells or ex vivo generated cells have been considered a possible approach (Levine, 2001).

Thus, there is a need for therapies that achieve the regeneration of the lymphoid lineages, in particular the regeneration of T cell-mediated immunity, in acquired immunodeficiencies or in tumor treatments that are accompanied with defects in the lymphoid system.

It was an object of the invention to investigate the molecular mechanisms involved in B-lineage commitment in order to harness these findings for human therapy.

In order to solve the object of the invention, it was sought to find out, by conditional inactivation of Pax5, whether B-lineage commitment requires only the transient activation or continuous expression of Pax5 during early B cell development.

The design of the experiments of the invention was based on the consideration that the inactivation of a commitment factor in an already committed cell could have at least three different phenotypic consequences: The factor-deprived cells may receive confusing signals and undergo cell death. Alternatively, these cells may remain irreversible committed to the selected lineage as evidenced by the transient role of Notch signaling in determining the glial cell fate of neural crest stem cells (Morrison et al., 2000). Finally, the cells may decommit and regain the broad developmental potential of an earlier progenitor cell.

The experiments of the invention have shown that the latter is the case for Pax5-dependent commitment to the B-lymphoid lineage. It was surprisingly found by conditional Pax5 inactivation in committed pro-B cells that Pax5 is required not only to initiate, but also to maintain its transcriptional program in early B cell development. It was found that, as a consequence of Pax5 inactivation, previously committed pro-B cells regain the capacity to differentiate into macrophages in vitro and to reconstitute T cell development in vivo in RAG2^{-/-} mice. Hence, the loss of Pax5 alone is sufficient to reverse B-lineage commitment by converting pro-B cells with a restricted B-lymphoid potential into hematopoietic progenitors with a broad developmental potential.

The findings of the present invention raise the intriguing possibility that wild-type pro-B cells have the ability to differentiate into other hematopoietic cell types by down-regulating Pax5 expression and thus reversing B cell commitment in response to external signals. In this context it is important to note that the CD19 gene is a direct Pax5 target (Kozmik et al., 1992), whose expression is entirely dependent on the presence of wild-type Pax5 protein levels (Nutt et al., 1997; Nutt et al., 1998). It was therefore argued that CD19 is not only an indicator of Pax5 activity (Nutt et al., 1999b), but also a decisive marker of B cell commitment (Nutt et al., 1999a; Nutt et al., 1999b). Surprisingly however, CD19⁺ progenitors, isolated from the bone marrow of adult mice, can differentiate upon exposure to appropriate cytokines into dendritic cells (Björk and Kincade, 1998) or macrophages (Montecino-Rodriguez et al., 2001), which was accompanied by down-regulation of CD19 expression (Björk and Kincade, 1998). These observations together with the data suggest that committed B cell progenitors can again broaden their developmental potential by repressing Pax5 transcription under physiological conditions.

Most importantly, the generation of multipotent hematopoietic progenitors from wild-type pro-B cells is also of medical relevance.

### BRIEF SUMMARY OF THE INVENTION

The present invention relates to pro-B cells of human origin that are deficient in functional Pax5 expression. (In the following, these cells are termed “Pax5-deficient pro-B cells”).

The Pax5-deficient pro-B cells of the invention have the capacity of long-term reconstitution, self-renewal and homing to the bone marrow. The Pax5-deficient pro-B cells of the invention have also a broad multilineage potential and the ability to completely restore T cell development.

In a further embodiment, the present invention relates to a method for producing human Pax5-deficient pro-B cells, comprising the steps of

a) isolating mononuclear cells from human tissue,

b) enriching the cell population obtained in a) for lymphoid progenitor cells,

c) in vitro differentiating the progenitor cells enriched in b) into pro-B cells and expanding the pro-B cells in culture with cytokines,

d) identifying and characterizing the pro-B cells according to the expression of B-lymphoid-specific cell surface proteins,

e) inactivating the Pax5 gene in the pro-B cells,

f) isolating and growing the Pax5-deficient pro-B cells under pro-B cell culture conditions.

In step a), for generating Pax5-deficient pro-B cells of human origin, suitable tissues, from which mononuclear cells can be isolated, are fetal liver, fetal cord blood, bone marrow and adult peripheral blood.

In step b), lymphoid progenitor cells can be enriched by sorting for cells co-expressing the antigens CD34, CD38 and CD10. The CD34⁺CD38⁺CD10⁺ lymphoid progenitor cells are isolated by state-of-the-art cell sorting methods using CyChrome-labeled anti-CD34 (e.g. 8G12), phycoerythrin-labeled anti-CD38 (e.g. T16) and fluorescein isothiocyanate-labeled anti-CD₁₀ (e.g. W8E7) antibodies, which can be obtained from commercial sources (Beckman-Coulter and Becton-Dickinson).

In step c), the sorted CD34⁺CD38⁺CD10⁺ lymphoid progenitors are expanded and differentiated in vitro, usually in co-culture with appropriate human or mouse stromal cells in a pro-B cell medium, an example of which is described in detail in Example 5. Examples for mouse stromal cells are: MS-5 cells (Berardi et al., 1997), ST2 cells (Ogawa et al., 1988) and S17 cells (Cumano et al., 1990).

The human cytokines that are used for pro-B cell culture to improve the expansion of human pro-B cells are usually present in recombinant form. They may be selected from stem cell factor (SCF), interleukin-2 (IL-2), interleukin-7 (IL-7), interleukin-15 (IL-15), Flt3 ligand (FL) and thymic stromal lymphopoietin (TSLP). These cytokines can be obtained from commercial sources (see Example 5), or if not commercially available, specially generated, e.g. recombinant human TSLP (Quentmeier et al., 2001). In a preferred embodiment, the cytokines are selected from IL-2 and/or IL-15 and/or SCF; usually, all three of these cytokines are present in the medium to expand pro-B cells. In an even more preferred embodiment, for further improvement, the cytokine mix contains additional cytokines, e.g. at least one of the cytokines IL-3, Flt3 ligand and TSLP. The pro-B cell medium contains at least three, preferably at least four cytokines.

In step d), the in vitro cultured pro-B cells are identified by their cell surface phenotype (CD34⁺CD19⁺CD10⁺CD79a⁺CD38⁻) by flow cytometric analysis using appropriately labeled anti-CD79a (e.g. J14.119) and anti-CD19 (e.g. HD37 antibodies) in combination with the anti-CD34, anti-CD10 and anti-CD38 antibodies described above.

In step e), the expression of the Pax5 gene is inactivated by antisense RNA, small interfering RNA or ribozyme strategies that are described in detail below.

In step f), the Pax5-deficient pro-B cells are identified by the loss of expression of one or more Pax5 target genes, preferably by loss of CD19 expression, which is known to be totally dependent on Pax5 function (Kozmik et al., 1992; Nutt et al., 1997). The CD34⁺CD19⁻CD10⁺CD79a⁺ pro-B cells lacking Pax5 expression are isolated by cell sorting and expanded in pro-B cell medium as described above under step (c).

As demonstrated in the experiments of the invention, murine Pax5-deficient pro-B cells can be obtained from committed pro-B cells in a two-step procedure. First, the pro B-cells are sorted from the bone marrow of Pax5^{F/F} CreED-30 mice and are then cultured on γ-irradiated stromal cells (ST2) in IL-7 containing IMDM medium. Second, following induction of Cre recombinase activity, the two floxed Pax5^{F} alleles are inactivated by Cre-mediated deletion of exon 2.

Human pro-B cells can be obtained in a similar manner as murine pro-B cells. The conditions required for the growth and culture of human cells, in particular the growth factors, can be determined empirically. Suitable culture conditions, including the growth factor mix, are described in Example 5. Conditions for culturing human pro-B cells, that are suitable for the method of the invention, have also been described by Wolf et al. (1991); Rawlings et al. (1995); Namikawa et al. (1996); Nishihara et al., (1998); Kurosaka et al. (1999); Barker et al. (2000) and Reynaud et al. (2003).

In order for the pro-B cells of the invention to achieve a therapeutic benefit, Pax5 function needs to be stably suppressed. To this end, in step e), the Pax5 function is preferably inactivated by one of the three methods described below.

To achieve long-term Pax5 inhibition, techniques are applied that ensure stable expression of inhibiting oligonucleotide molecules. The strategies interfering with Pax5 function employ synthetic oligonucleotides capable of hybridizing with DNA or RNA. A preferred embodiment of the invention involves the application of RNA interference (RNAi). RNAi is the process of sequence-specific post-transcriptional gene silencing initiated by double-stranded RNA that is homologous in sequence to the silenced gene. Small interfering RNA (siRNA) duplexes of 21 to 22 nucleotides were shown to be a new powerful tool for inhibiting gene function in mammalian cells (Elbashir et al., 2001). Sui et al. (2002) and Brummelkamp et al. (2002a, 2002b) have recently reported vector-based systems for stable expression of short interfering RNAs. These systems are based on a vector, in which a synthetic, gene-specific target sequence encoding the siRNA is expressed under the control of a promoter that is suitable for transcription of small, non-coding RNA. The siRNAs are thus produced from the vector following its introduction into mammalian cells by standard transfection (e.g. electroporation, lipofection) or viral infection protocols (e.g. retroviral infection).

As an alternative to siRNA, antisense oligonucleotides can be used to interfere with the expression of the native Pax5 protein.

Antisense oligonucleotides are short stretches of nucleotides that are complementary to a region of the target mRNA and can specifically suppress expression of that particular transcript. Examples of antisense oligonucleotides and their use in experimental and clinical settings have been recently reviewed (Braasch and Corey, 2002; Agrawal et al., 1998; Galderisi et al., 1999; Gewirtz, 1998). The antisense nucleic acid can take the form of RNA expressed from a vector, which has been transfected into the cell or take the form of a DNA or RNA oligonucleotide which can be introduced into cells through a variety of means, e.g. by means of cationic liposomes, cationic porphyrins, fusogenic peptides, and artificial virosomes, or cell permeabilization with streptolysin-O and electroporation. Cationic lipids form stable complexes with oligonucleotides, which exhibit improved cellular uptake (Bennett et al., 1992; Lappalainen et al., 1994), thus resulting in enhanced antisense activity.

Alternatively, Pax5 can be inactivated by means of ribozymes. Similar to antisense oligonucleotides, ribozymes bind to substrate RNA through Watson-Crick base pairing, which leads to sequence-specific cleavage of transcripts. Two types of ribozymes, the hammerhead ribozyme and the hairpin ribozyme, have been extensively studied due to their small size and rapid kinetics. Their application has been recently reviewed in several publications (Hampel, 1998; Vaish et al., 1998; Birikh et al., 1997; Earnshaw and Gait, 1997; Kore and Eckstein, 1999).

In the present invention, ribozymes can be imported into the cell by various means, as described above for antisense oligonucleotides, or they can be expressed from a vector, which offers the advantage of continued intracellular production of these molecules (Irie et al., 1999; Smith et al., 1997).

Preferably, the Pax5-inhibiting oligonucleotide molecules are produced from a viral vector. For this purpose, the lentiviral vectors are most preferred, as they have been shown to successfully infect primary cells (Luther-Wyrsch et al., 2001). Furthermore, lentiviral vectors have already proven to be suitable for in vivo gene therapy applications due to the following characteristics (Buchschacher et al., 2000; VandenDriessche et al., 2002):

Since lentiviruses are stably integrated into chromosomal DNA and have little tendency to be epigenetically silenced, they offer the potential for long-term expression. Moreover, lentiviral vectors can efficiently transduce dividing as well as non-dividing cells. The ability to pseudotype lentiviral vectors with the vesicular stomatitis virus G (VSV-G) protein allows for the production of high-titer stocks of stable virus with a broad host range (Emi et al., 1991).

To achieve Pax5 inhibition, expression cassettes encoding Pax5-inhibiting oligonucleotide molecules are inserted into lentiviral vectors (Lever, 1996; Follenzi et al., 2002). These vectors are then transfected by standard methods (e.g. electroporation, lipofection) into specialized packaging cells (Kafri et al., 1999) to generate pseudotyped lentiviruses for infection of human pro-B cells that have been cultured as described in step (c) and Example 5.

Based on the gene function-inhibitory methods described above and the published sequence of the human Pax5 gene (Adams et al., 1992), the person skilled in the art can determine the Pax5 siRNA, antisense or ribozyme target sequences and construct vectors for transfection or infection of pro-B cells.

In another embodiment, the present invention relates to Pax5-deficient pro B cells for use in the therapy of disorders associated with a depletion of the lymphoid system, in particular in the treatment of immunodeficiencies, e.g. AIDS and in the therapy of tumors.

For therapeutic applications, either autologous Pax5-deficient pro-B cells are used or Pax5-deficient pro-B cells that are derived from HLA-matched donors (LeBien, 2000).

During progression of AIDS, the bone marrow becomes pancytopenic possibly due to excessive synthesis of cytokines that interfere with the survival and/or generation of progenitors. Essentially, the bone marrow is massively reduced in cellularity due to constant fever and infections. By repopulating the bone marrow with HLA-matched or autologous Pax5-deficient pro-B cells, the break-down of the lymphoid system (T, NK and DC cells) can be alleviated or partially prevented. Repopulation is achieved after intravenous injection of a single dose of Pax5-deficient pro-B cells in an amount that is sufficient to at least partially restore T cell immunity, which can be monitored by an increase of mature T cells in the peripheral blood. Depending on the severity of the condition and on the body weight of the patient, the dosage usually comprises approximately 10⁷ to 10⁸ cells suspended in isotonic saline solution. Initially, treatment of the patient with antibiotics and immunosuppressive drugs is advisable to avoid possible side effects of the treatment.

The application of Pax5-deficient pro-B cells is also useful in tumor therapies. Chemotherapy of tumor patients often leads to massive side effects in the hematopoietic system. The cell numbers of various different lineages are massively reduced. In cancer therapy, Pax5-deficient pro-B cells can be used for transient counteraction of iatrogenic effects during tumor therapy. Injection of Pax5-deficient pro-B cells is beneficial for the rapid recovery of the lymphoid system until the endogenous HSC itself is recovering from the insult.

Alternatively, Pax5-deficient pro-B cells can be used in non-myeloablative stem cell therapy.

Non-myeloablative therapy has recently been developed as a less toxic alternative to myeloablation, the use of which is limited because of severe acute adverse effects. Non-myeloablative therapy was developed on the theory that abolition of abnormal cells may not solely depend on prior myeloablation by high-dose irradiation therapy. It has been suggested that engraftment of donor cells may also help to destroy malignant cells in the recipient. It is argued that conditioning regimens need not be myeloablative, provided that patients receive enough immunosuppressive treatment to allow donor engraftment. Non-myeloablative therapy was, inter alia, successfully applied for metastatic kidney cell carcinoma (Childs et al., 1999; Childs et al., 2000). In this therapeutic approach, bone marrow cells (enriched for stem cells) of an HLA-matched donor are injected into tumor patient who is irradiated at a low dose (approximately one sixth of the lethal dose) prior to cell injection. Four weeks later, most bone marrow cells of the patient are of donor origin. As HLA matching is never perfect, some minor loci have not been perfectly matched. These minor loci are considered to be responsible for a graft-versus-tumor reaction that may lead to the rejection of the tumor.

As an alternative to the currently used protocols that use HLA-matched bone marrow cells enriched for stem cells, the Pax5-deficient pro-B cells of the invention may be applied.

Non-myeloablative therapy may be associated with severe adverse effects, because it relies on immunosuppressive treatment to prevent graft-versus-host disease after transplantation. Such treatment predisposes recipients to infection and may also reduce the anti-malignancy effects of donor cells. Although these negative effects are considered to be acceptable because they are outweighed by the benefits of preserving host marrow function until donor marrow engrafts, the application of Pax5-deficient cells in non-myeloablative therapy provides the additional advantage to alleviate these effects.

In a further embodiment, the invention relates to Pax5-deficient pro-B cells that are modified in such a way that, in addition to Pax5, other functions are either partially suppressed or completely inactivated. The modification of the cells may also be such that one or more gene functions are activated.

In a preferred embodiment, the Pax5-deficient pro-B cells are modified in order to prevent HIV entry or intracellular HIV propagation.

HIV-1 infection of susceptible cells is mediated by the specific interaction of viral envelope glycoproteins with the cell surface CD4 receptor and a chemokine coreceptor, CCR5 or CXCR4. Since CCR5 has been shown to be a non-essential co-receptor, it is considered an ideal function to be targeted for preventing HIV infection.

Hence, in a preferred embodiment, the Pax5-deficient pro-B cells are additionally made defective in CCR5 expression.

Based on the published CCR5 sequence (see e.g. Raport et al., 1996), CCR5 expression can be inhibited in a similar manner as described above for the suppression of Pax5 function, e.g. by siRNAs, antisense oligonucleotides or ribozymes. Pax5 function and CCR5 function may be suppressed by introducing two different inhibiting constructs into pro-B cells, e.g. RNA inhibiting molecules targeted to Pax5 on the one hand and to CCR5 on the other hand, or they may be co-repressed by designing a construct encoding both Pax5- and CCR5-inhibiting RNA molecules and importing this molecule into pro-B cells according to one of the methods described above for the inhibition of Pax5 function.

By way of example, a construct containing an anti-CCR5 ribozyme heterotrimer targeted to three different cleavage sites in CCR5 mRNA can be introduced into the Pax5-deficient pro-B cells by retroviral vectors as described by Bai et al. (2001). These authors have shown that cells stably transduced with an anti-CCR5 heterotrimer showed a marked reduction in the surface expression of CCR5 and a concomitant 70% reduction in macrophage-tropic viral infection.

In addition to sequences inhibiting CCR5 co-receptor function, the RNA-inhibiting constructs, e.g. ribozymes, may carry anti-viral sequences, e.g. anti-HIV-1 tat-rev sequences, as described by Bai et al. (2001). Ribozyme heterodimers carrying such sequences in addition to anti-CCR5 sequences showed a similar inhibition of CCR5 surface expression and reduced infectability. It was shown that trimeric and multimeric ribozyme constructs, when transduced into CD34+hematopoietic progenitor cells, conferred resistance to HIV-1 infection (Bai et al., 2001).

In a further embodiment, the invention relates to a pharmaceutical composition containing Pax5-deficient pro-B cells of human origin. The Pax5-deficient cells can be formulated according to methods known in the art, e.g. as known for stem cell therapy. By way of example, autologous Pax5-deficient pro-B cells or Pax5-deficient pro-B cells derived from a donor that is HLA-matched to the patient, are suspended in isotonic saline solution.

### DESCRIPTION OF THE FIGURES

FIGS. 1A-D: Gene expression changes in pro-B cells following Pax5 inactivation. (A) Loss of CD19 expression upon Cre-mediated deletion of Pax5. Pax5^{F/F} CreED-30 pro-B cells were analysed by flow cytometry with PE-anti-CD19 and APC-anti-B220 antibodies either before (F/F) or 5 weeks after treatment (Δ/Δ) with 4-hydroxy-tamoxifen (OHT; 1 μM). (B) Deletion kinetics. The loss of the floxed exon 2 of Pax5 was monitored by PCR analysis of DNA prepared from OHT-treated Pax5^{F/F} CreED-30 pro-B cells. (C) Loss of Pax5 protein. Whole cell lysates of OHT-treated Pax5^{F/F} CreED-30 pro-B cells were analysed by sequential Western blotting with polyclonal anti-Pax5 and monoclonal anti-tubulin antibodies. (D) Semiquantitative RT-PCR analysis. Transcripts of the indicated genes were analysed by RT-PCR at the indicated days after OHT-mediated induction of Cre activity. PCR products were visualised on agarose gels by ethidium bromide staining. “FL” denotes the full-length transcript of the Pax5^{F} allele and “ΔE2” denotes the Pax5^{Δ} transcript lacking exon 2. rEST1, rEST2 and rEST3 refer to Pax5-repressed (r) transcripts of unknown function that were identified by cDNA microarray analysis. The sequences of the μₘ and δₘ transcripts were amplified from the third or first constant exon to the first transmembrane exon, respectively. Hence, the 5′ end of these transcripts has not been determined and could correspond to the Iμ, μ or Dμ transcript.

FIGS. 2A-C: In vitro differentiation of Pax5^{Δ/Δ} pro-B cells into macrophages. (A) Flow cytometric analysis. Pax5^{Δ/Δ} pro-B cells were cultured for 18 days on ST2 cells in the presence or absence of IL-7. (B) May-Grünwald-Giemsa staining of cytospin preparations of Pax5^{Δ/Δ} pro-B cells that were grown for 3 weeks on ST2 cells with or without IL-7. (C) Phagocytosis. In vitro differentiated Pax5^{Δ/Δ} macrophages were incubated with FITC-labelled E. coli, while the nuclei were stained with DAPI.

FIGS. 3A-C: Pax5^{Δ/Δ} pro-B cells reconstitute T cell development in RAG2^{-/-} mice. (A) Thymus reconstitution by Pax5^{Δ/Δ} pro-B cells. 10⁶ Pax5^{Δ/Δ} pro-B cells were injected into 550 rad-irradiated RAG2^{-/-} mice. Three weeks after transfer, the thymus consisted of 80×10⁶ cells (compared to 5×10⁶ cells of uninjected control RAG2^{-/-} mice). The CD4⁺CD8⁺ double-positive (DP), CD4⁺ and CD8⁺ single-positive (SP) thymocytes were analyzed by three-color flow cytometry using FITC-anti-CD4, PE-anti-CD8 and APC-anti-TCRβ antibodies. The three subpopulations (gated on CD4/CD8 expression) exhibited the expected levels of TCRβ expression. Stippled lines indicate the absence of TCRβ □staining in control RAG2^{-/-} thymocytes. (B) Failure of Pax5^{Δ/Δ} pro-B cells to differentiate into B cells. The spleen of the same injected RAG2^{-/-} mouse shown in (A) was analysed by flow cytometry using APC-anti-B220 and PE-anti-IgM antibodies. (C) Commitment of Pax5^{F/F} pro-B cells to the B-lymphoid lineage. B220⁺ pro-B cells were sorted from the bone marrow of Pax5^{F/F} mice and cultured in vitro for only 2 weeks prior to injection of 5×10⁵ cells into 550 rad-irradiated RAG2^{-/-} mice. After 3 weeks, the thymus and spleen were analyzed by flow cytometry. The Thy1.2⁺TCRβ⁻ cells in the thymus correspond to the pro-T cells of the RAG2^{-/-} host.

FIGS. 4A-F: Pax5^{+/-} pro-B cells decommit upon switching of expression from the wild-type to the targeted Pax5 allele. (A) Generation of CD19⁻ Pax5^{+/-} pro-B cell clones. CD19⁺ pro-B cells were isolated from the bone marrow of Pax5^{+/-} mice and cultured in the presence of ST2 cells and IL-7 for 8 weeks (w) prior to the generation of single-cell clones by FACS sorting. The pool and a derived clone of Pax5^{+/-} pro-B cells was analyzed by flow cytometry with PE-anti-CD19 and APC-anti-B220 antibodies at the indicated times after the start of in vitro culture. The cloned Pax5^{+/-} pro-B cells were separately analyzed for β-galactosidase activity after loading with the fluorogenic substrate CMFDG. The stippled line indicates wild-type pro-B cells that were similarly treated with CMFDG. (B) Semiquantitative RT-PCR analysis. Expression of the indicated genes was determined by RT-PCR in wild-type (+/+), homozygous (−/−) and heterozygous (+/−) Pax5 mutant pro-B cells. The pool (p) and a clone (c) of Pax5^{+/-} pro-B cells were analyzed after 1 and 13 weeks (w) of in vitro culture, respectively. (C,D) In vitro differentiation of CD19⁻ Pax5^{+/-} pro-B cells into macrophages. Cloned CD19⁻ Pax5^{+/-} pro-B cells were cultured for 3 weeks on ST2 cells in the presence or absence of IL-7 and then analyzed by flow cytometry with PE-anti-B220 and APC-anti-Mac-1 antibodies (C). The phagocytic activity of the differentiated cells was measured by incubation with FITC-labeled E. coli (D). (E) Thymus reconstitution by CD19⁻ Pax5^{+/-} pro-B cells. The pro-B cells of a CD19⁻ Pax5^{+/-} clone were infected with a retrovirus expressing a truncated human (h) CD2 protein and then injected into 550 rad-irradiated RAG2^{-/-} mice. Three weeks after transfer, the thymus was analyzed by flow cytometry with PE-anti-hCD2, FITC-anti-CD4 and APC-anti-CD8 antibodies. The expression of CD4 and CD8 is shown for the hCD2⁺ thymocytes of donor origin. (F) Long-term engraftment of injected CD19⁻ Pax5^{+/-} pro-B cells in the bone marrow of RAG2^{-/-} mice. Eleven weeks after cell transfer, the bone marrow was analyzed by flow cytometry with PE-anti-hCD2, FITC-anti-CD19 and APC-anti-B220 antibodies. The expression CD19 and B220 is displayed for the hCD2⁺ cell of donor origin.

### DETAILED DESCRIPTION OF THE INVENTION

If not otherwise stated, the following materials and methods were used in the Examples:

Transgenic mice.

Pax5^{+/-}, Pax5^{F/F}, RAG2^{-/-} and CreED-30 mice were maintained on the C57BL/6x129/Sv background and genotyped as described (Horcher et al., 2001; Urbanek et al., 1994; Schwenk et al., 1998; Shinkai et al., 1992).

Pro-B Cell Culture.

B220⁺ pro-B cells were sorted from the bone marrow of Pax5^{+/-} or Pax5^{F/F} CreED-30 mice and cultured on y-irradiated ST2 cells in IL-7 containing IMDM medium as described (Nutt et al., 1997). Pax5^{+/-} pro-B cell clones were generated by sorting single cells from a CD19⁻ Pax5^{+/-} pro-B cell pool with a FACSVantage TSO flow-cytometer (Becton-Dickinson).

Antibodies and Flow Cytometry.

Single-cell suspensions were stained and analysed on a FACSCalibur flow-cytometer (Becton-Dickinson) as described (Urbanek et al., 1994). The following fluorescein isothiocyanate (FITC)-, phycoerythrin (PE)- or allophycocyanin (APC)-labelled antibodies were purchased from PharMingen (San Diego, Calif.): anti-B220 (RA3-6B2), anti-CD4 (L3T4), anti-CD8 (53-6.7), anti-CD19 (1D3), anti-Mac-i (M1/70), anti-IgM (M41.42), anti-TCRβ (H57-597) and anti-human CD2 (RPA-2.10) antibodies. The PE-anti-F4/80 (C1:A3-1) antibody were obtained from Serotec (Oxford, England). β-Galactosidase activity was measured by loading the pro-B cells with the fluorogenic substrate 5-chloromethylfluorescein di-β-D-galactopyranoside (CMFDG, Molecular Probes) as described (Nutt et al., 1999c).

Kinetic Analysis of Cre-Mediated Pax5 Inactivation.

B220⁺ pro-B cells were sorted from the bone marrow of 4-week-old Pax5^{F/F} CreED-30 mice, expanded in vitro and then treated with 1 μM 4-hydroxy-tamoxifen (OHT; Research Biochemicals International, Natick, Mass.) for 3 days followed by subsequent culturing in the absence of OHT. The CD19⁻ Pax5^{Δ/Δ} pro-B cells were purified to homogeneity by eliminating the few non-deleting CD19⁺ Pax5^{F/F} pro-B cells by FACS sorting. The kinetic experiments were performed with freshly isolated Pax5^{F/F} CreED-30 pro-B cells that were expanded ex vivo to 30×10⁶ cells for only 9 days prior to OHT treatment as described above. Pro-B cells (6.5×10⁶) were withdrawn at daily intervals after OHT addition. DNA isolated from 0.5×10⁶ cells was analysed by PCR genotyping (Horcher et al., 2001) to confirm deletion of Pax5 exon 2. Total RNA was prepared from 5×10⁶ cells, using the Trizol Reagent (Gibco-BRL, Life Technologies). Reverse transcription (with random hexamer primers) and semiquantitative PCR were performed as described (Horcher et al., 2001; Nutt et al., 1999c).

The following primers (5′-3′) and PCR conditions were used:

| gene | SEQ ID NO: | annealing ° C. | cycles | size (bp) |
|---|---|---|---|---|
| Igδₘ | 1 and 2 | 55 | 25 | 595 |
| Igμₘ | 3 and 4 | 55 | 25 | 513 |
| J-chain | 5 and 6 | 55 | 30 | 338 |
| Pax5 | 7 and 8 | 55 | 25 | 593 (FL) |
| | | | | 427 (ΔE2) |
| HPRT | 9 and 10 | 55 | 25 | 313 |
| EBF | 11 and 12 | 55 | 30 | 481 |
| M-CSF-R | 13 and 14 | 57 | 30 | 1200 |
| CD19 | 15 and 16 | 59 | 25 | 847 |
| mb-1 | 17 and 18 | 59 | 30 | 820 |
| B29 1 | 19 and 20 | 59 | 30 | 243 |
| CIITA | 21 and 22 | 59 | 30 | 764 |
| rEST1 | 23 and 24 | 57 | 25 | 492 |
| rEST2 | 25 and 26 | 57 | 25 | 680 |
| rEST3 | 27 and 28 | 57 | 30 | 595 |
| Pax5-lacZ | 29 and 30 | 55 | 25 | 611 |

The PCR products were separated on agarose gels and visualized by ethidium bromide staining. Protein samples were prepared by directly dissolving and boiling 1×10⁶ cells in 80 μl of 2×SDS sample buffer. Total protein (10 μl) was separated by 10% SDS-PAGE and analyzed by Western blotting with a rabbit polyclonal antibody directed against the Pax5 paired domain (Adams et al., 1992).

In Vitro Macrophage Differentiation.

Pax5^{Δ/Δ} pro-B cells were cultured on ST2 cells in the absence of IL-7 for ˜10 days followed by terminal differentiation in IMDM medium containing M-CSF (25 ng/ml; R&D Systems). The phagocytic activity of differentiated cells was determined by incubation with FITC-labelled heat-inactivated E. coli (Molecular Probes) as described (Nutt et al., 1999a).

Pro-B Cell Transfer into RAG2^{-/-} Mice.

RAG2^{-/-} mice at 8-12 weeks of age were γ-irradiated with 550 rad and intravenously injected with 1-5×10⁶ in vitro cultured Pax5^{Δ/Δ}, Pax5^{F/F} or Pax5^{+/-} pro-B cells as described (Rolink et al., 1999). Prior to injection, the Pax5^{+/-} pro-B cells were infected with the pMI retrovirus expressing a human CD2 protein lacking its intracellular domain (Deftos et al., 1998).

### EXAMPLES

### Example 1

### B-Lineage Commitment Requires Continuous Expression OF PAX5 During Early B Cell Development

To address the question whether B-lineage commitment requires only the transient activation or continuous expression of Pax5 during early B cell development, the inventors took advantage of the Cre-loxP system for conditional inactivation of a floxed (F) Pax5^{F} allele carrying loxP sequences on either side of the paired domain exon 2 (Horcher et al., 2001). B cell development was normal in Pax5^{F/F} mice, whereas Pax5^{Δ/Δ} mice, generated by germline deletion (Δ) of exon 2 (Horcher et al., 2001), were phenotypically indistinguishable from Pax5^{-/-} mice (Urbanek et al., 1994).

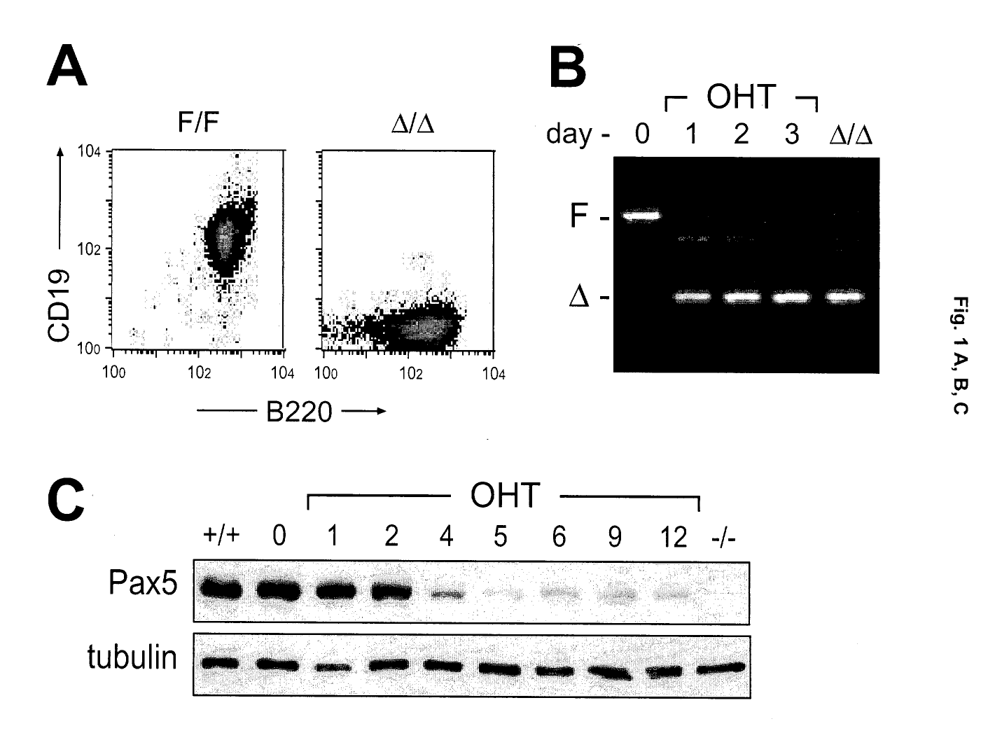

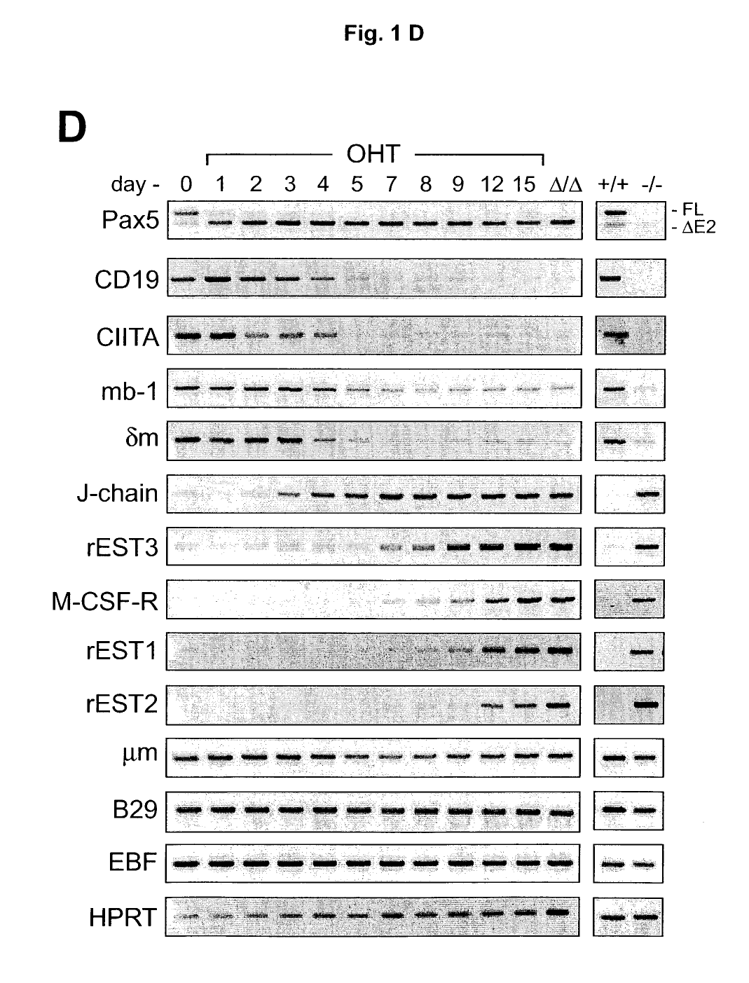

For conditional Pax5 inactivation at the pro-B cell stage, Pax5^{F/F} mice were crossed with the transgenic line CreED-30, which expresses the Cre-EBD[G521R] fusion protein under the control of the immunoglobulin Eμ enhancer and SV40 promoter in the B-lymphoid lineage (Schwenk et al., 1998). The Cre-EBD[G521R] protein consists of the Cre recombinase linked to a mutant human oestrogen receptor ligand-binding domain (EBD) that is insensitive to the natural ligand β-oestradiol, but is responsive to the synthetic antagonist 4-hydroxy-tamoxifen (OHT). This posttranslational induction system allows the specific induction of Cre recombinase activity in B-lymphocytes by OHT treatment (Schwenk et al., 1998). Therefore pro-B cells were sorted from the bone marrow of Pax5^{F/F} CreED-30 mice and cultured these cells in vitro on stromal cells in the presence of IL-7. Flow cytometric analysis revealed that these pro-B cells express the B cell markers B220 and CD19 (FIG. 1A, F/F). As transcription of the target gene CD19 is strictly dependent on Pax5 function (Nutt et al., 1997; Nutt et al., 1998), these data demonstrate that the CD19⁺ Pax5^{F/F} pro-B cells express normal levels of Pax5 and have thus undergone commitment to the B-lymphoid lineage (for further arguments, see below). Treatment of these pro-B cells with 1 μM OHT resulted in the specific loss of CD19 expression, indicating that the CreED-30 transgene can be used for efficient inactivation of the floxed Pax5 allele under the in vitro culture conditions used (FIG. 1A, Δ/Δ). It is important to note that the proliferation of the pro-B cells remained largely unaffected by the OHT treatment (data not shown). Hence, the OHT-induced inactivation of Pax5 did result neither in a significant cell loss nor in the outgrowth of a minor subpopulation of Pax5^{Δ/Δ} pro-B cells.

Time course analyses revealed that Pax5^{F/F} CreED-30 pro-B cells efficiently deleted the Pax5 exon 2 within 1 day of OHT treatment (FIG. 1B). Within the same short period, the pro-B cells predominantly expressed the truncated Pax5 transcript lacking exon 2 (FIG. 1D) in agreement with the fact that the Pax5 mRNA has a short half-life of ˜2 hours (Nutt et al., 1999c). In contrast, the more stable Pax5 protein was reduced to a low level only 4 days after OHT addition (FIG. 1C). Next used RT-PCR analysis was used to investigate the gene expression changes caused by Pax5 inactivation. One class consisted of the control genes, encoding the ubiquitous HPRT mRNA and the B-cell-specific EBF, B29 (Igβ) and μₘ transcripts, which were neither differentially expressed in wild-type and Pax5^{-/-} pro-B cells nor affected by conditional Pax5 loss (FIG. 1D). In contrast, the expression of target genes, which are normally activated by Pax5 in pro-B cells, started to decline at day 4, concomitant with the loss of Pax5 protein (FIG. 1D). These target genes code for the cell surface protein CD19 and Igα (mb-1) (Nutt et al., 1998), the transcription factor CIITA (Horcher et al., 2001) and the alternatively spliced δₘ transcript of the immunoglobulin IgH gene (Horcher et al., 2001). Conversely, the genes that are normally silenced by Pax5 at the onset of B cell development were derepressed upon Pax5 inactivation (FIG. 1D). This class of genes codes for the secreted J-chain (Rinkenberger et al., 1996), the M-CSF receptor (Nutt et al., 1999a) and transcripts of unknown function. Hence, the expression pattern of Pax5^{F/F} pro-B cells appears to be indistinguishable from that of Pax5^{+/+} pro-B cells, but can be converted by Pax5 inactivation into that of Pax5^{-/-} pro-B cells. It was therefore concluded that the Pax5-dependent gene expression program is reversible in pro-B cells undergoing Pax5 inactivation.

### Example 2

### PAX5-Deficient PRO-B Cells Adopt Again the Broad Developmental Potential of Early Hematopoietic Progenitor Cells

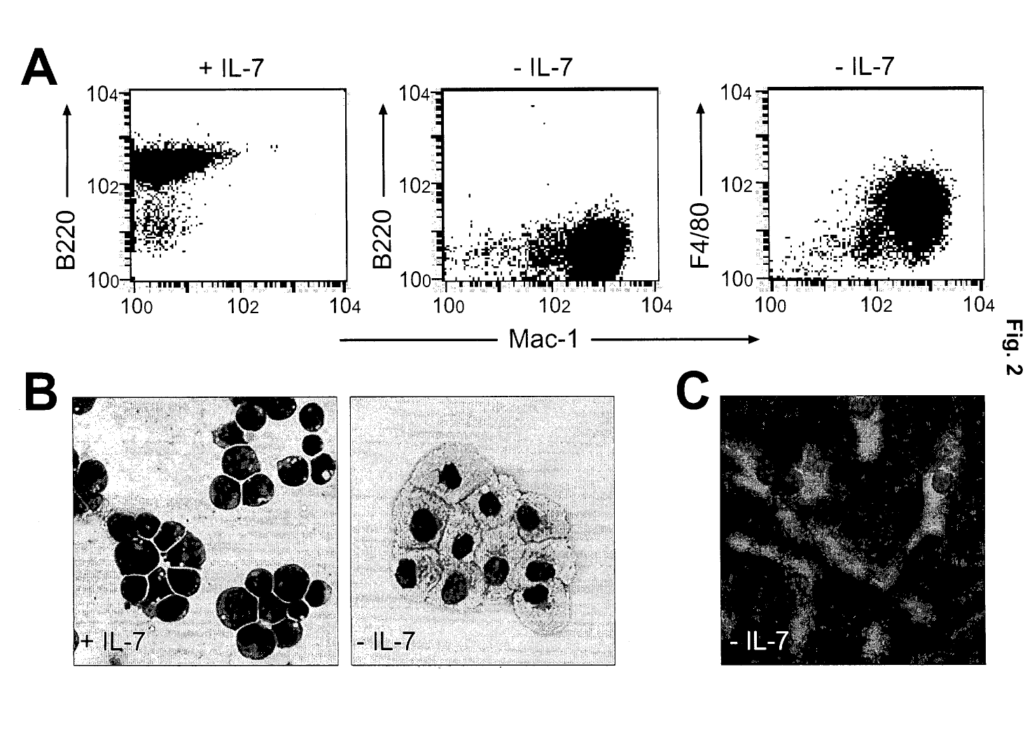

As the Pax5^{Δ/Δ} pro-B cells were directly derived from committed Pax5^{F/F} pro-B cells, it was next investigated whether the Pax4^{Δ/Δ} pro-B cells remained committed to the B cell pathway or adopted again the broad developmental potential of early hematopoietic progenitor cells. The potential of the Pax5^{Δ/Δ} pro-B cells to differentiate into macrophages was assessed by culturing them in the absence of IL-7 on stromal ST2 cells, which abundantly produce the myeloid cytokine M-CSF (Nutt et al., 1999a). Pax5^{Δ/Δ} cells, that were differentiated under these conditions for up to 3 weeks, down-regulated the B cell surface protein B220, expressed the macrophage markers Mac-1 and F4/80 (FIG. 2A) and displayed the characteristic morphology of enlarged vacuolar macrophages (FIG. 2B). These differentiated cells furthermore phagocytosed fluorescently labeled E. coli (FIG. 2C), indicating that the Pax5^{Δ/Δ} pro-B cells can differentiate into mature macrophages like the Pax5^{-/-} pro-B cells (Nutt et al., 1999a).

### Example 3

### PAX5 Deficient PRO-B Cells have the Potential to Develop into T-Lymphocytes

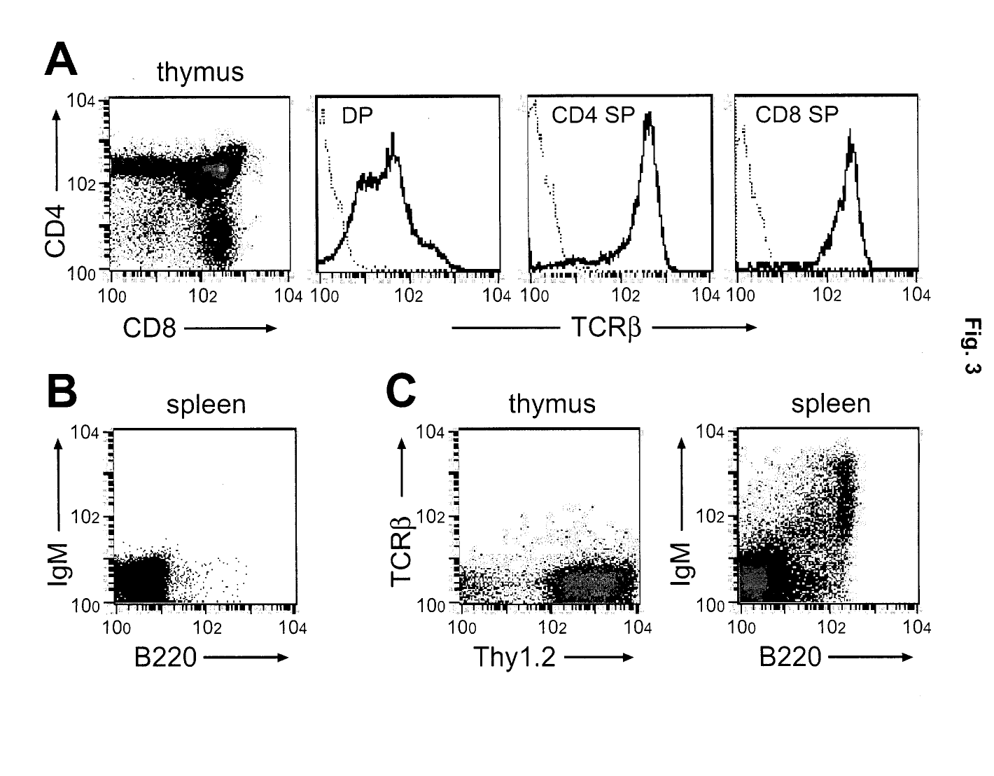

The potential of Pax5^{Δ/Δ} pro-B cells to develop into T-lymphocytes was analyzed by intravenous injection into sublethally irradiated RAG2^{-/-} mice, which have an early block in T and B cell development due to the absence of V(D)J recombination (Shinkai et al., 1992). Three weeks after cell transfer, the cellularity of the thymus was considerably increased due to the presence of CD4⁺CD8⁺(double-positive) as well as CD4⁺CD8⁻ and CD8⁺CD4⁻ (single-positive) thymocytes (FIG. 3A). These thymocyte subpopulations were derived from the injected Pax5^{Δ/Δ} pro-B cells, as they expressed the T cell receptor (TCR) β similar to wild-type thymocytes (FIG. 3A). Hence, the Pax5^{Δ/Δ} pro-B cells could fully reconstitute T cell development, but failed to generate IgM⁺ B cells in the spleen of RAG2^{-/-} mice (FIG. 3B) in analogy to Pax5^{-/-} pro-B cells (Rolink et al., 1999). The opposite situation was observed for the Pax5^{F/F} pro-B cells, which failed to give rise to TCRβ⁺thymocytes, but developed into IgM⁺ B cells in the spleen of injected RAG2^{-/-} mice (FIG. 3C). Hence, the Pax5^{F/F} pro-B cells are fully committed to the B cell pathway. It was therefore concluded that the inactivation of Pax5 alone is sufficient to revert the narrow B-lymphoid potential of Pax5^{F/F} pro-B cells to the broad multilineage potential characteristic of early hematopoietic progenitor cells.

### Example 4

### Decommitment of PAX5^{+/-} PRO-B Cells upon Switching of Expression from the Wild-Type to the Targeted PAX5 Allele

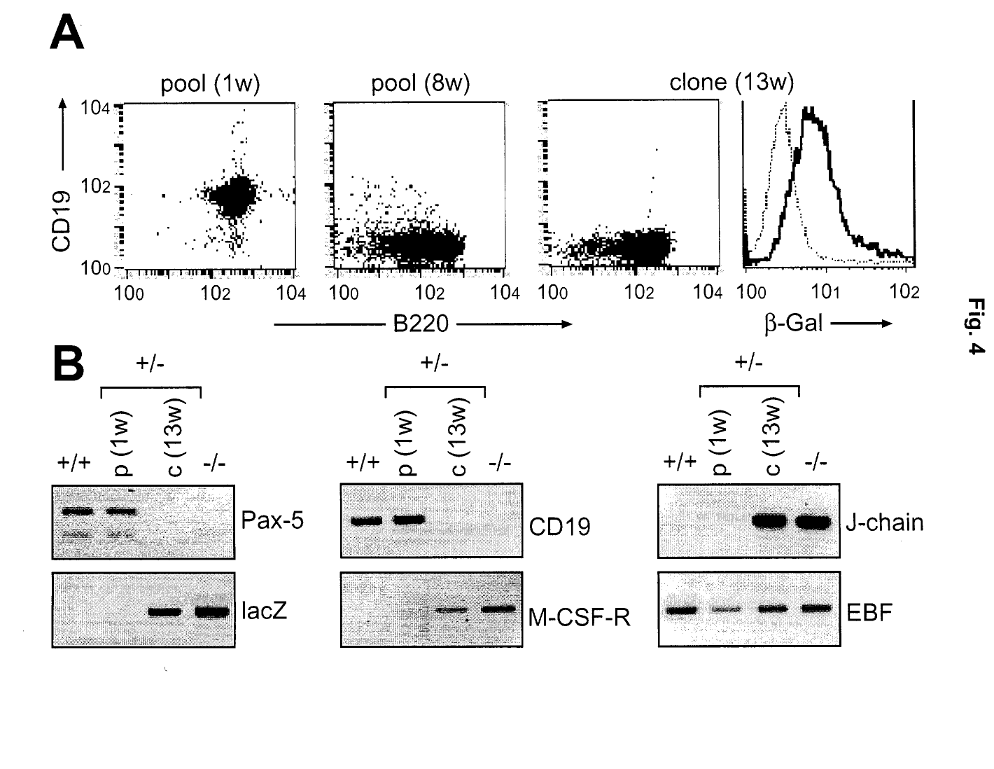

The heterozygous Pax5^{+/-} mouse provided a second opportunity to investigate the reversibility of Pax5-dependent B-lineage commitment, as the pro-B cells of this mouse preferentially express only one of the two Pax5 alleles (Nutt et al., 1999c). Interestingly, heterozygous CD19⁺ pro-B cells are able to switch expression from the wild-type Pax5 allele to the targeted Pax5^{lacZ} (−) allele during in vitro culture, which may reflect a proliferative advantage of the Pax5 null phenotype for the in vitro propagation of pro-B cells (Nutt et al., 1999b; Nutt et al., 1999c). This allele switching results in the loss of expression of the Pax5 target gene CD19 and simultaneous activation of the lacZ gene of the targeted Pax5 locus (Nutt et al., 1999b; Nutt et al., 1999c) (FIG. 4A). Therefore, CD19⁻ β-Gal⁺ pro-B cell clones were derived from Pax5^{+/-} bone marrow (FIG. 4A) and demonstrated by RT-PCR analysis that these pro-B cells switched off the expression of activated Pax5 target genes and derepressed genes normally suppressed by Pax5 (FIG. 4B; data not shown). Hence, the CD19⁻ β-Gal⁺ pro-B cells apparently reversed their gene expression program to that of homozygous Pax5^{-/-} cells, although, as genotypically heterozygous cells, they still have the ability to reactivate the wild-type Pax5 allele. These CD19⁻ Pax5^{+/-} pro-B cells were also decommitted, as they differentiated, upon IL-7 withdrawal into functional Mac-1⁺F4/80⁺ macrophages with high phagocytic activity, similar to Pax5^{-/-} cells (FIGS. 4C,D; data not shown). To study the T-lymphoid potential, the CD19⁻ Pax5^{+/-} pro-B cells were first infected with a human (h) CD2-expressing retrovirus prior to injection into sublethally irradiated RAG2^{-/-} mice. Three weeks after cell transfer, the thymus contained CD4⁺CD8⁺, CD4⁺CD8⁻ and CD8⁺CD4⁻ T cells of donor origin (hCD2⁺), indicating that the CD19⁻ Pax5^{+/-} pro-B cells are also able to reconstitute T cell development (FIG. 4E). Thymic reconstitution was still observed 11 weeks after cell transfer due to long-term engraftment of the CD19⁻B220⁺ Pax5^{+/-} pro-B cells in the bone marrow, from where these progenitors continuously seed the thymus (FIG. 4F, data not shown). In addition to these CD19^{-B}220⁺ pro-B cells, the bone marrow contained CD19⁺B220⁺ B-lymphocytes of donor origin (hCD2⁺), which have re-entered the B cell pathway due to activation of the wild-type Pax5 allele (FIG. 4F). Finally, the bone marrow cells also contained a third hCD2⁺CD19⁻B220⁻ cell population (FIG. 4F), which primarily consisted of mature CD4⁺ and CD8⁺ T-lymphocytes (data not shown). In summary, heterozygous Pax5^{+/-} pro-B cells did not only regain the broad developmental potential of early hematopoietic progenitors upon switching to the mutant Pax5 allele, but at the same time maintained their competence to undergo B cell development by reactivating the wild-type Pax5 allele in vivo.

### Example 5

### Culture of Human PRO-B Cells

Low-density mononuclear cells are prepared from human umbilical cord blood and enriched for CD34⁺ progenitor cells by MACS sorting with CD34 immunomagnetic beads (Miltenyi, Auburn, Calif.). The bead-sorted CD34⁺ cells (40-80% pure) are then stained with CyChrome-labeled anti-CD34, fluorescein isothiocyanate-coupled anti-CD10 and phycoerythrin-labeled anti-CD19 antibodies, and the CD34⁺CD19⁻CD10⁻ cells are sorted using a FACSVantage TSO flow-cytometer (Becton-Dickinson). The purified CD34⁺CD19⁻CD10⁻ cells are cultured in a pro-B cell medium on Petri dishes that are precoated with a confluent γ-irradiated monolayer of murine MS-5 stromal cells (Berardi et al, 1997). The pro-B cell medium consists of Iscove's Dulbecco modified medium (IMDM) containing 10% human AB serum (Institut Jacques BOY, Reims, France), 5% pre-tested fetal calf serum (HCC-6400; Stem Cell Technologies Inc., Vancouver, Canada) and the following recombinant (r) human (hu) cytokines: rhu-stem cell factor (SCF, 50 ng/ml; Amgen, Calif., USA), rhu-interleukin (IL)-2 (5 ng/ml, Diaclone, France) and rhu-IL-15 (1 ng/ml; Diacone, France). The pro-B cells are cultured at 37° C. in 5% CO₂ and fed every week by exchanging half of the medium. To improve the expansion of human B cells, serial experiments are conducted by adding the cytokines IL-7 and/or Flt 3 ligand (both cytokines are obtained from R&D Systems) and/or TSLP (recombinantly produced) to the culture medium in varying concentrations ranging from 2-10 ng/ml. Suitable concentrations are 5 ng/ml for both IL-7 and Flt ligand, and 7.5 ng/ml for TSLP, respectively.

### REFERENCES

Adams, B., et al., Genes Dev. 6, 1589-1607 (1992).

Agrawal, S., and Zhao, Q., Curr. Opin. Chem. Biol. 2, 519-528 (1998).

Bai, J., Rossi, J., and Akkina, R., AIDS Res. Hum. Retroviruses 17, 385-399 (2001).

Bain, G., et al., Cell 79, 885-892 (1994).

Barker, J., and Verfaillie, C. M., Leukemia 14, 1614-1620 (2000).

Bennett, C. F., Chiang, M. Y., Chan, H., Shoemaker, J. E., and Mirabelli, C. K., Mol. Pharmacol. 41, 1023-1033 (1992).

Berardi, A. C., Meffre, E., Pflumio, F., Katz, A., Vainchenker, W., Schiff, C., and Coulombel, L., Blood 89, 3554-3564 (1997).

Birikh, K. R., Heaton, P. A., and Eckstein, F., Eur. J. Biochem. 245, 1-16 (1997).

Björk, P., and Kincade, P. W., J. Immunol. 161, 5795-5799 (1998).

Braasch, D. A., and Corey, D. R., Biochemistry 41, 4503-4510 (2002).

Brekken et al., Cancer Research 60, 5117-5124 (2000).

Brummelkamp, T. R., Bernards, R., and Agami, R., Science 296, 550-553 (2002a).

Brummelkamp, T. R., Bernards, R., and Agami, R., Cancer Cell 2, 243-247 (2002b).

Buchschacher, G. L. Jr., and Wong-Staal, F., Blood 95, 2499-2504 (2000).

Busslinger, M., Nutt, S. L., and Rolink, A. G., Curr. Opin. Immunol. 12, 151-158 (2000).

Capaccioli, S., Di Pasquale, G., Mini, E., et al., Biochem. Biophys. Res. Commun. 197, 818-825 (1993).

Childs, R. W., et al., J. Clinical Oncology 17, 2044-2049 (1999).

Childs, R., Chernoff, A., Contentin, N., Bahceci, E., Schrump, D., Leitman, S., Read, E. J., Tisdale, J., Dunbar, C., Linehan, W. M., Young, N. S., and Barrett, A. J., N. Engl. J. Med. 343, 750-758 (2000).

Cumano, A., Dorshkind, K., Gillis, S., and Paige, C. J., Eur. J. Immunol. 20, 2183-2189 (1990).

Deftos, M. L., He, Y.-W., Ojala, E. W., and Bevan, M. J., Immunity 9, 777-786 (1998).

Drevs et al., Cancer Research 60, 4819-4824 (2000).

Earnshaw, D. J., and Gait, M. J., Antisense Nucleic Acid Drug Dev. 7, 403-411 (1997).

Elbashir, S. M., et al. Nature 411, 494-498 (2001).

Emi, N., Friedmann, T., and Yee, J. K., J. Virol. 65, 1202-1207 (1991).

Fischer, A., et al., Annu. Rev. Immunol. 15, 93-124 (1997).

Follenzi, A., and Naldini, L., Methods Enzymol. 346, 454-465 (2002).

Galderisi, U., Cascino, A., and Giordano, A., J. Cell Physiol. 181, 251-257 (1999).

Gewirtz, A. M., Curr. Opin. Hematol. 5, 59-71 (1998).

Hampel, A., Prog. Nucleic Acid Res. Mol. Biol. 5, 1-39 (1998).

Hennequin et al., J. Med. Chem. 42, 5369-5389 (1999).

Horcher, M., Souabni, A., and Busslinger, M., Immunity 14, 779-790 (2001).

Irie, A., Anderegg, B., Kashani-Sabet, M., et al., Antisense Nucleic Acid Drug Dev. 9, 341-349 (1999).

Kafri, T., van Praag, H., Ouyang, L., Gage, F. H., and Verma, I. M., J. Virol. 73, 576-584 (1999).

Kee, B. L., and Murre, C., J. Exp. Med. 188, 699-713 (1998).

Kondo, M., Weissman, I. L., and Akashi, K., Cell 91, 661-672 (1997).

Kore, A. R., and Eckstein, F., Biochemistry 38, 10915-10918 (1999).

Kozmik, Z., Wang, S., Dörfler, P., Adams, B., and Busslinger, M., Mol. Cell. Biol. 12, 2662-2672 (1992).

Kurosaka, D., LeBien, T. W., and Pribyl, J. A. R., Exp. Hematol. 27, 1271-1281, (1999).

LeBien, T. W., Blood 96, 9-23 (2000).

Lever, A. M., Gene Ther. 3, 470-471 (1996).

Levine, B. L., Mosca, J. D., Riley, J. L, et al., Science 272,1939-1943 (1996).

Levine, A. M., Scadden, D. T., Zaia, J. A., and Krishnan, A., Hematology (Am. Soc. Hematol. Educ. Program.), 463-478 (2001).

Lin, H. and Grosschedl, R., Nature 376, 263-267 (1995).

Lin et al., Cell Growth Diff. 9, 49-58, (1998).

Luther-Wyrsch, A., et al., Hum. Gene Ther. 12, 377-389 (2001).

Mitsuyasu, R. T., Anton, P. A., Deeks, S. G., et al., Blood 96, 785-793 (2000).

Montecino-Rodriguez E., Leathers H., and Dorshkind K., Nature Immunol. 2, 83-88 (2001).

Morrison, S. J., et al., Cell 101, 499-510 (2000).

Naldini, L., Blomer, U., Gallay, P., Ory, D., Mulligan, R., Gage, F. H., Verma, I. M. and Trono, D., Science 272, 263-267 (1996).

Namikawa, R., Muench, M. O., de Vries, J. E., and Roncarolo, M. G., Blood 87, 1881-1890, (1996)

Nishihara, M., Wada, Y., Ogami, K., Ebihara, Y., Ishii, T., Tsuji, K., Ueno, H., Asano, S., Nakahata, T., and Maekawa, T., Eur. J. Immunol. 28, 855-864 (1998).

Nutt, S. L., Urbanek, P., Rolink, A., and Busslinger, M., Genes Dev. 11, 476-491 (1997).

Nutt S. L., Morrison A. M., Dörfler P., Rolink A., and Busslinger M., EMBO J. 17, 2319-2333 (1998).

Nutt, S. L., Heavey, B., Rolink, A. G., and Busslinger, M., Nature 401, 556-562 (1999a).

Nutt, S. L., Rolink, A. G., and Busslinger, M., Cold Spring Harb. Symp. Quant. Biol. 64, 51-59 (1999b).

Nutt, S. L., et al., Nature Genet. 21, 390-395 (1999c).

Ogawa, M., Nishikawa, S., Ikuta, K., Yamamura, F., Naito, M., Takahashi, K., and Nishikawa, S.-I., EMBO J. 7, 1337-1343 (1988).

Poznansky, M. C., Evans, R. H., Foxall, R. B., et al., Nat. Biotechnol. 18, 729-734 (2000).

Presta et al., Cancer Research 57, 4593-4599 (1997).

Quentmeier, H., Drexler, H. G., Fleckenstein, D., Zaborski, M., Armstrong, A., Sims, J. E., and Lyman, S. D., Leukemia 15, 1286-1292 (2001).

Raport, C. J., Gosling, J., Schweickart, V. L., Gray, P. W. and Charo, I. F., J. Biol. Chem. 271, 17161-17166 (1996).

Rawlings, D. J., Quan, S. G., Kato, R. M., and Witte, O. N., Proc. Natl. Acad. Sci. USA 92, 1570-1574 (1995).

Reynaud, D., Lefort, N., Manic, E., Coulombel, L., and Levy, Y., Blood, prepublished online January 30 (2003).

Rinkenberger, J. L., Wallin, J. J., Johnson, K. W., and Koshland, M. E., Immunity 5, 377-386 (1996).

Rolink, A. G., Nutt, S. L., Melchers, F., and Busslinger, M., Nature 401, 603-606 (1999).

Rosenberg, E. S., Billingsley, J. M., Caliendo, A. M., et al., Science 278, 1447-1450 (1997).

Saleh et al., Cancer Research 56, 393-401 (1996).

Schwenk, F., Kühn, R., Angrand, P.-O., Rajewsky, K., and Stewart, A. F., Nucleic Acids Res. 26, 1427-1432 (1998).

Shinkai, Y., et al., Cell 68, 855-867 (1992).

Sigvardsson, M., O'Riordan, M., and Grosschedl, R., Immunity 7, 25-36 (1997).

Smith, S: M., Maldarelli, F., and Jeang, K. T., J. Virol. 71, 9713-9721 (1997).

Sui, G., Soohoo, C., Affar, E. B., Gay, F., Shi, Y., Forrester, W. C., and Shi, Y., Proc Natl Acad Sci USA 99, 5515-5520 (2002).

Urbánek, P., Wang, Z.-Q., Fetka, I., Wagner, E. F., and Busslinger, M., Cell 79, 901-912 (1994).

Vaish, N. K., Kore, A. R., and Eckstein, F., Nucleic Acids Res. 26, 5237-5242 (1998).

VandenDriessche, T., Naldini, L., Collen, D., and Chuah, M. K., Methods Enzymol. 346, 573-589 (2002).

Weissman, I. L., Cell 100, 157-168 (2000).

Wolf, M. L., Buckley, J. A., Goldfarb, A., Law, C. L., and LeBien, T., J. Immunol. 147, 3324-3330 (1991).

Yang, O. O., Tran, A. C., Kalams, S. A., Johnson, R. P., Roberts, M. R., and Walker, B. D., Proc Natl Acad Sci USA. 94, 11478-11484 (1997).

Zhuang, Y., Soriano, P., and Weintraub, H., Cell 79, 875-884 (1994).

The entire disclosure of each document cited (including patents, patent applications, journal articles, abstracts, laboratory manuals, books, entries in sequence databases, or other disclosures) in this application is hereby incorporated herein by reference.

What is claimed is:

1. Pro-B cells of human origin that are deficient in functional Pax5 expression.

2. A method for producing human Pax5-deficient pro-B cells of claim 1, comprising the steps of
a) isolating mononuclear cells from human tissue,
b) enriching the cell population obtained in a) for lymphoid progenitor cells,
c) in vitro differentiating these progenitors into pro-B cells and expanding the pro-B cells in culture with cytokines,
d) identifying and characterizing the pro-B cells according to the expression of B-lymphoid-specific cell surface proteins,
e) inactivating the Pax5 gene in the cultured pro-B cells,
f) isolating and growing the Pax5-deficient pro-B cells under pro-B cell culture conditions.

3. The method of claim 2, wherein in step a) the tissue is selected from fetal liver, fetal cord blood, bone marrow and adult peripheral blood.

4. The method of claim 2, wherein in step b) lymphoid progenitor cells are enriched by sorting for cells co-expressing the antigens CD34, CD38 and CD10.

5. The method of claim 2, wherein in step c) the sorted CD34⁺CD38⁺CD10⁺ lymphoid progenitors are expanded and differentiated in vitro in co-culture with appropriate stromal cells in a pro-B cell medium.

6. The method of claims 2, wherein the pro-B cell are expanded with recombinant human cytokines.

7. The method of claim 6, wherein the pro-B cells are expanded with the cytokines stem cell factor (SCF) and/or interleukin-2 (IL-2) and/or interleukin-15 (IL-15).

8. The method of claim 7, wherein the pro-B cells are expanded with stem cell factor (SCF), interleukin-2 (IL-2) and interleukin-15 (IL-15).

9. The method of claim 7 or 8, wherein the culture medium contains, in addition, the cytokines thymic stromal lymphopoietin (TSLP) and/or interleukin-7 (IL-7) and/or Flt-3 ligand.

10. The method of claim 9, wherein the culture medium contains thymic stromal lymphopoietin (TSLP), interleukin-7 (IL-7) and Flt-3 ligand.

11. The method of claim 2, wherein in step d) pro-B cells are identified by their expression of CD34⁺CD19⁺CD10⁺CD79a⁺CD38⁻.

12. The method of claim 11, wherein the pro-B cells are sorted by flow cytometric analysis using labeled anti-CD79a, anti-CD19, anti-CD34, anti-CD10 and anti-CD38 antibodies.

13. The method of claim 2, wherein in step e) expression of the Pax5 gene is inactivated by means of antisense RNA.

14. The method of claim 2, wherein in step e) expression of the Pax5 gene is inactivated by means of small interfering RNA.

15. The method of claim 2, wherein in step e) expression of the Pax5 gene is inactivated by means of a ribozyme.

16. The method of any one of claims 10 to 15, wherein
i. an expression cassette encoding one or more Pax5-inhibiting oligonucleotide molecules is inserted into lentiviral vector,
ii. the lentiviral vector is transfected into specialized packaging cells to generate pseudotyped lentiviruses,
iii. pro-B cells obtained in step (c) are infected with the pseudotyped lentivirus obtained in (ii.) and cultured.

17. The method of claim 2, wherein in step f) the Pax5-deficient pro-B cells are identified by loss of expression of one or more Pax5 target genes.

18. The method of claim 1, wherein the Pax5-deficient pro-B cells are identified by loss of CD19 expression.

19. The method of claim 2, wherein pro-B cells lacking Pax5 expression are isolated by cell sorting and expanded in pro-B cell medium as in step c).

20. Autologous human Pax5-deficient pro-B cells or Pax5-deficient pro-B cells derived from a HLA-matched donor for use in the therapy of disorders associated with a depletion of the lymphoid system.

21. Autologous human Pax5-deficient pro-B cells or Pax5-deficient pro-B cells derived from a HLA-matched donor for the treatment of immunodeficiencies.

22. Autologous human Pax5-deficient pro-B cells or Pax5-deficient pro-B cells derived from a HLA-matched donor for the treatment of AIDS.

23. Pax5-deficient pro-B cells derived from a HLA-matched donor for use in the therapy of tumors.

24. Pro-B cells derived from a HLA-matched donor for use in non-myeloablative tumor therapy.

25. Pharmaceutical composition containing Pax5-deficient pro-B cells of human origin.

26. Pax5-deficient pro-B cells in which, in addition to Pax5, other functions are either partially suppressed or completely inactivated.
